(19) United States
(12) Patent Application Publication
(10) Pub. No.: US 2023/0093352 A1
(43) Pub. Date: Mar. 23, 2023

(54) Sexed Sperm Bulk Separation Systems

(71) Applicant: Cytolutions, LLC, Columbus, OH (US)

(72) Inventor: Kristie Krug, Upper Arlington, OH (US)

(73) Assignee: Cytolutions, LLC, Columbus, OH (US)

(21) Appl. No.: 17/480,322

(22) Filed: Sep. 21, 2021

(51) Int. Cl. G01N 33/569; C12Q 1/24

(52) U.S. Cl. CPC G01N 33/56966; C12Q 1/24

(57) ABSTRACT

A broad object of a the instant invention can be to provide a method for separating X and Y sperm cells within a sample sperm cell population, the method including (i) differentiating between and (ii) separating sperm cells that have undergone a cellular process and sperm cells that have not undergone the cellular process, whereby a majority of the sperm cells that have undergone the cellular process can comprise one of X or Y sperm cells, and a majority of the sperm cells that have not undergone the cellular process can comprise the other of X or Y sperm cells. As to particular embodiments, the cellular process can be a maturational step. As to particular embodiments, the maturational step can be capacitation. As to particular embodiments, the maturational step can be the acrosome reaction. As to particular embodiments, non-viable and viable sperm cells can also be (i) differentiated between and (ii) separated.

### I. SUMMARY OF THE INVENTION

A broad object of a the instant invention can be to provide a method for separating X and Y sperm cells within a sample sperm cell population, the method including (i) differentiating between and (ii) separating sperm cells that have undergone a cellular process and sperm cells that have not undergone the cellular process, whereby a majority of the sperm cells that have undergone the cellular process can comprise one of X or Y sperm cells, and a majority of the sperm cells that have not undergone the cellular process can comprise the other of X or Y sperm cells. As to particular embodiments, the cellular process can be a maturational step. As to particular embodiments, the maturational step can be capacitation. As to particular embodiments, the maturational step can be the acrosome reaction. As to particular embodiments, non-viable and viable sperm cells can also be (i) differentiated between and (ii) separated.

Naturally, further objects of the invention are disclosed throughout other areas of the specification, drawings, and claims.

### II. BRIEF DESCRIPTION OF THE DRAWINGS

FIG. 1 illustrates a particular embodiment of the instant method.

FIG. 2 illustrates a particular embodiment of the instant method.

FIG. 3 illustrates a particular embodiment of the instant method.

FIG. 4 illustrates a particular embodiment of the instant method.

FIG. 5 illustrates a particular embodiment of the instant method.

FIG. 6 shows flow cytometry analysis of an ejaculate sample before differentiation and separation, whereby the sample was stained with Hoechst to determine its X sperm cell and Y sperm cell content.

FIG. 7 shows flow cytometry analysis of Sample A after a 3 hour incubation in a capacitation-inducing medium, and subsequent differentiation and separation of (i) non-viable sperm cells, (ii) capacitated sperm cells, and (iii) acrosome-reacted sperm cells, whereby the sample was stained with Hoechst to determine its X sperm cell and Y sperm cell content.

FIG. 8 shows flow cytometry analysis of Sample B after a 5 hour incubation in a capacitation-inducing medium, and subsequent differentiation and separation of (i) non-viable sperm cells, and (ii) capacitated sperm cells, whereby the sample was stained with Hoechst to determine its X sperm cell and Y sperm cell content.

FIG. 9 shows flow cytometry analysis of Sample C after a 24 hour incubation in a capacitation-inducing medium, and subsequent differentiation and separation of (i) non-viable sperm cells, (ii) capacitated sperm cells, and (iii) acrosome-reacted sperm cells, whereby the sample was stained with Hoechst to determine its X sperm cell and Y sperm cell content.

### III. DETAILED DESCRIPTION OF THE INVENTION

A sperm (also called spermatozoon, plural spermatozoa) is a male germ cell capable of fertilizing an egg, whereby a sperm cell, in addition to having autosomes, carries genetic information for determining the sex of the offspring, and specifically either an X chromosome (and correspondingly is an X chromosome-bearing sperm cell, herein referred to as an “X sperm cell”) or a Y chromosome (and correspondingly is a Y chromosome-bearing sperm cell, herein referred to as a “Y sperm cell”). The present invention provides a method for separating X sperm cells and Y sperm cells in a sample sperm cell population comprising both and thus, can be a method for sex-selection of sperm cells. Significantly, following separation, at least some of the desired subpopulation of sperm cells can be viable and able to fertilize an egg to produce offspring.

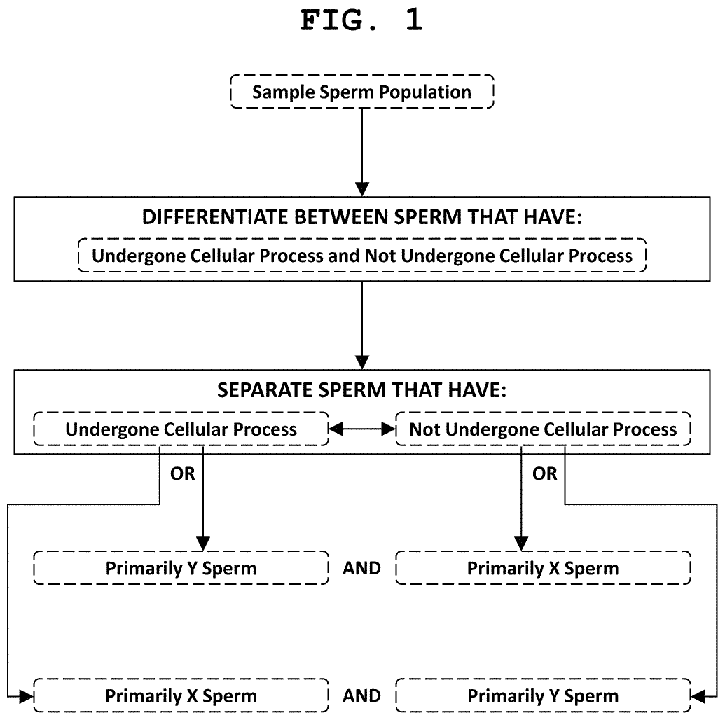

Now referring primarily to FIG. 1, in more detail, the instant method includes (i) differentiating between sperm cells that have undergone a cellular process and sperm cells that have not undergone the cellular process, and (ii) separating the sperm cells that have undergone the cellular process and the sperm cells that have not undergone the cellular process.

Significantly, as to particular embodiments, a majority of the sperm cells that have undergone the cellular process can comprise or consist of one of X sperm cells or Y sperm cells, and a majority of the sperm cells that have not undergone the cellular process can comprise or consist of the other of X sperm cells or Y sperm cells.

As used herein, the term “undergone” means to experience and/or to be subjected to, whether partially (meaning at least initiated) or completely.

As used herein, the term “majority” means the greater quantity and/or a number or percentage equaling more than half of the total.

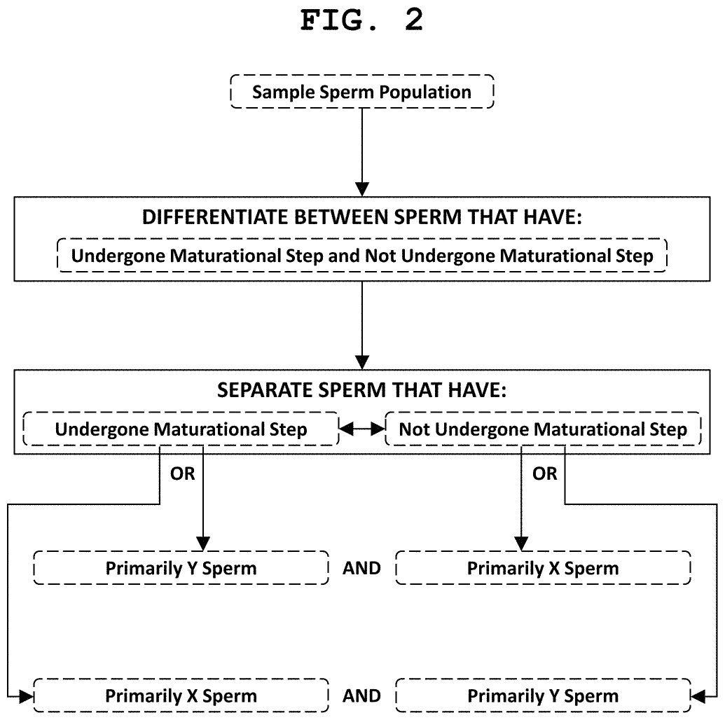

Now referring primarily to FIG. 2, as to particular embodiments of the instant method, the cellular process can be a maturational step; correspondingly, the method can include (i) differentiating between sperm cells that have undergone a maturational step and sperm cells that have not undergone the maturational step, and (ii) separating the sperm cells that have undergone the maturational step and the sperm cells that have not undergone the maturational step. Relevantly, a majority of the sperm cells that have undergone the maturational step can comprise or consist of one of X sperm cells or Y sperm cells, and a majority of the sperm cells that have not undergone the maturational step can comprise or consist of the other of X sperm cells or Y sperm cells.

### Capacitation Induction

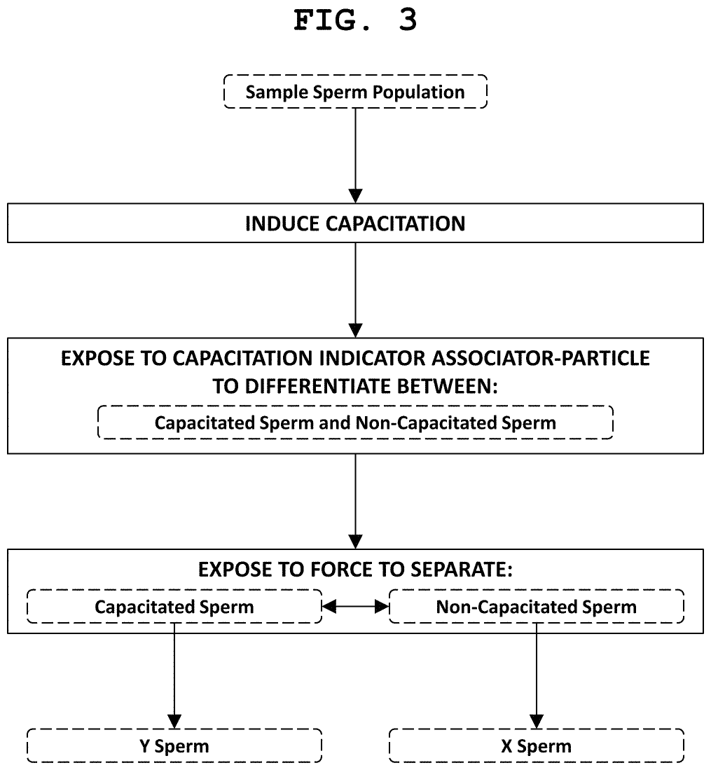

Now referring primarily to FIG. 3, as to particular embodiments of the instant method, separation of X sperm cells and Y sperm cells can be facilitated by differentiating between sperm cells that have undergone capacitation (hence capacitated sperm cells) and sperm cells that have not undergone capacitation (hence non-capacitated sperm cells), whereby said capacitation can be a cellular process and/or a maturational step which results in an increased permeability of the plasma membrane to Ca′ ions, and culminates in the ability of a sperm cell to acrosome-react upon coming into contact with the zona pellucida and then fuse with the egg's plasma membrane to ultimately fertilize the egg.

As per the present invention, the rate of capacitation can differ between X sperm cells and Y sperm cells, whereby as used herein “rate of capacitation” can mean the percent of sperm cells that have at least initiated and/or completed capacitation over time or per unit of time. In particular, Y sperm cells can undergo capacitation more quickly (or faster) than X sperm cells, and this difference in capacitation rate can be exploited to separate X sperm cells and Y sperm cells. Thus, as to particular embodiments of the instant method, separation of X sperm cells and Y sperm cells can be facilitated by differentiating between sperm cells that undergo capacitation more quickly (expectedly Y sperm cells) and sperm cells that undergo capacitation more slowly (expectedly X sperm cells).

Typically, in vivo, capacitation takes place when ejaculated semen comes into contact with the female genital tract. In vitro, capacitation can occur naturally or be induced and/or triggered.

As to particular embodiments, the instant method can include (i) differentiating between and (ii) separating sperm cells that have undergone capacitation naturally in vitro.

Again referring primarily to FIG. 3, as to other particular embodiments of the instant method, separation of X sperm cells and Y sperm cells can be facilitated by inducing capacitation or changes that may be associated with capacitation to differentiate between capacitated sperm cells and non-capacitated sperm cells, whereby the instant method exploits the premise that Y sperm cells can undergo capacitation more quickly than X sperm cells. Accordingly, as to particular embodiments, the instant method can include inducing capacitation to generate a capacitation-induced sperm cell subpopulation in which capacitation can be induced in at least a portion of the Y sperm cells within the sample sperm cell population.

As to particular embodiments of the instant method, capacitation can be associated with and/or induced by a change in pH.

As to particular embodiments of the instant method, capacitation can be associated with and/or induced by an increase in pH.

As to particular embodiments of the instant method, capacitation can be associated with and/or induced by an increase in external pH (pHₑ), which can be the pH of the environment, typically a medium or media, in which the sperm cells are contained.

As to particular embodiments of the instant method, capacitation can be associated with and/or induced by an increase in pHₑ of at least about 0.36 pH units from the initial external pH, herein referred to as a “baseline pHₑ.” Without being bound by any particular theory, an increase in pHₑ of at least about 0.36 pH units from the baseline pHₑ may be necessary for the removal of sialic acid groups from the surface of the sperm cell, whereby sialic acids (also referred to as neuraminic acids) can occupy terminal positions of oligosaccharides in glycosylated proteins (or glycoproteins), providing negatively-charged points of interaction.

It may be understood that the exposed sialic acid content of X sperm cells can be greater (or higher) than that of Y sperm cells, and correspondingly, the exposed sialic acid content of Y sperm cells can be lesser (or lower) than that of X sperm cells. Said another way, X sperm cells can have more (or a greater amount) of cell surface sialic acid groups than Y sperm cells. Following and again, without being bound by any particular theory, it may be that Y sperm cells can undergo capacitation more quickly than X sperm cells because Y sperm cells can have a lesser amount of exposed cell surface sialic acid groups which may need to be removed and/or cleaved for capacitation to occur.

As to particular embodiments of the instant method, capacitation can be induced by increasing pHₑ by at least about 0.36 pH units from the baseline pHₑ via a medium, herein referred to as a “capacitation-inducing medium”; correspondingly, the method can include exposing the sample sperm cell population to a capacitation-inducing medium to generate a capacitation-induced sperm cell subpopulation in which capacitation can be induced in at least a portion of the Y sperm cells within the sample sperm cell population.

To achieve an increase in pHₑ of at least about 0.36 pH units, as to particular embodiments, the instant method can include determining the baseline pHₑ, which can facilitate proper selection of a capacitation-inducing medium that will increase the baseline pHₑ by at least about 0.36 pH units, whereby such a capacitation-inducing medium can be a herein be referred to as a “selected capacitation-inducing medium.” This step may be important because the baseline pHₑ of sample sperm cell populations can vary significantly, for example based upon the source of the sample sperm cell population and/or its storage conditions (for example, fresh or frozen-thawed) prior to subjection to the instant method.

As illustrative examples, capacitation-inducing media which may be useful with the instant method can include Tyrode's albumin lactate pyruvate (TALP) medium/buffer, Sp-TALP medium/buffer, TRIS medium/buffer, bovine gamete medium 1 (BGM1), or the like, or combinations thereof. Of course, for use with the instant method, the medium can be pH-adjusted to increase the determined baseline pHₑ by at least about 0.36 pH units.

Regarding a fresh sample sperm cell population, the method can include determining the baseline pHₑ. Subsequent to measuring the baseline pHₑ, the method can include removing the seminal plasma from the sample sperm cell population. Following, the method can include selecting a capacitation-inducing medium based on the determined baseline pHₑ to generate a selected capacitation-inducing medium. The method can consequently include exposing the sample sperm cell population to the selected capacitation-inducing medium to induce capacitation of at least a portion of the Y sperm cells within the sample sperm cell population.

Regarding a frozen-thawed sample sperm cell population, the method can include determining the baseline pHₑ prior to combining the sperm cells with a freezing-appropriate medium, as this medium may have a pHₑ which differs from the baseline pHₑ. Following, the method can include selecting a capacitation-inducing medium based on the determined baseline pHₑ to generate a selected capacitation-inducing medium. After thawing, the method can subsequently include exposing the sample sperm cell population to the selected capacitation-inducing medium to induce capacitation of at least a portion of the Y sperm cells within the sample sperm cell population.

To elaborate on exposure, the sample sperm cell population can be exposed to the selected capacitation-inducing medium for a period of time, whereby said period of time can be sufficient to induce capacitation of at least a portion of the Y sperm cells within the sample sperm cell population. During exposure, the combined sample sperm cell population and selected capacitation-inducing medium can be maintained at a selected temperature, for example in an incubator at about 37° C. in about 5% CO₂.

As to particular embodiments, the period of time can be in a range of between about 30 minutes to about 24 hours. As to particular embodiments, the period of time can be: less than about 24 hours, less than about 23 hours, less than about 22 hours, less than about 21 hours, less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, or less than about 1 hours.

As to particular embodiments, the selected temperature can be in a range of: between about room temperature to about 40° Celsius, between about room temperature to about 37° Celsius, between about 20° Celsius to about 40° Celsius, or between about 20° Celsius to about 37° Celsius. As to particular embodiments, the selected temperature can be about room temperature. As to particular embodiments, the selected temperature can be about 20° Celsius. As to particular embodiments, the selected temperature can be about 37° Celsius.

As to particular embodiments of the instant method, it can be that a greater increase in pHₑ from the baseline pHₑ can induce capacitation more quickly, which may result in a decrease in the period of time needed for exposure to the selected capacitation-inducing medium.

As to particular embodiments of the instant method, induction of capacitation can be associated with heparin; following, the sample sperm cell population can be exposed to heparin.

As to particular embodiments, to enhance capacitation induction, heparin can be added to the selected capacitation-inducing medium.

As to other particular embodiments, heparin alone, meaning without a medium which increases pHₑ by at least about 0.36 pH units from the baseline pHₑ, can be used to induce capacitation.

As to particular embodiments of the instant method, induction of capacitation can be associated with caffeine; following, the sample sperm cell population can be exposed to caffeine.

As to particular embodiments, to enhance capacitation induction, caffeine can be added to the selected capacitation-inducing medium.

As to other particular embodiments, caffeine alone, meaning without a medium which increases pHₑ by at least about 0.36 pH units from the baseline pHₑ, can be used to induce capacitation.

### Capacitated Sperm Cell Differentiation

As per the present invention, capacitation can result in the gain and/or exposure of a targetable molecule(s) which can subsequently serve as a “capacitation indicator” or a biomarker indicative (i) of at least the initiation of capacitation and/or (ii) that capacitation has been completed. Accordingly, capacitated sperm cells can comprise a capacitation indicator whereas non-capacitated sperm cells can be void of such a capacitation indicator. As a result, the instant method can include employing and/or using a capacitation indicator which can be associated with capacitated sperm cells to differentiate the capacitated sperm cells from non-capacitated sperm cells.

As to particular embodiments, the instant method can include associating a capacitation indicator associator with a capacitation indicator to differentiate capacitated sperm cells from non-capacitated sperm cells.

As an illustrative example, as per the present invention, in association with capacitation, there can be a loss of sialic acid groups from the surface of the sperm cell. This loss can unmask binding sites for lectins, which are proteins or glycoproteins that have specific affinities for particular saccharide molecules, whereby such a saccharide can serve as a capacitation indicator, and a lectin which associates with said saccharide can serve as a capacitation indicator associator.

As a first example of a capacitation indicator associator, peanut agglutinin (PNA) can have a strong specificity for disaccharides with terminal galactose. As a second example of a capacitation indicator associator, concanavalin A (ConA) can recognize mannose and glucose. As a third example of a capacitation indicator associator, Pisum sativum agglutinin (PSA) can display specificity toward mannose and glucose. As a fourth example of a capacitation indicator associator, Anguilla anguilla agglutinin (AAA) can recognize fucose.

Correspondingly, as to particular embodiments of the instant method, separation of X sperm cells and Y sperm cells can be facilitated by differentiating sperm cells that have undergone capacitation (expectedly primarily Y sperm cells) from sperm cells that have not undergone capacitation (expectedly primarily X sperm cells) by selecting for sperm cells comprising a capacitation indicator which a capacitation indicator associator associates with, for example via binding to.

Thus, as to particular embodiments of the instant method, separation of X sperm cells and Y sperm cells can be facilitated by differentiating sperm cells (i) with which PNA associates and/or (ii) to which PNA binds (hence capacitated sperm cells, and expectedly primarily Y sperm cells) from sperm cells that PNA does not associate with and/or bind to (hence non-capacitated sperm cells, and expectedly primarily X sperm cells).

Additionally, as to particular embodiments of the instant method, separation of X sperm cells and Y sperm cells can be facilitated by differentiating sperm cells (i) with which ConA associates and/or (ii) to which ConA binds (hence capacitated sperm cells, and expectedly primarily Y sperm cells) from sperm cells that ConA does not associate with and/or bind to (hence non-capacitated sperm cells, and expectedly primarily X sperm cells).

Further, as to particular embodiments of the instant method, separation of X sperm cells and Y sperm cells can be facilitated by differentiating sperm cells (i) with which PSA associates and/or (ii) to which PSA binds (hence capacitated sperm cells, and expectedly primarily Y sperm cells) from sperm cells that PSA does not associate with and/or bind to (hence non-capacitated sperm cells, and expectedly primarily X sperm cells).

Moreover, as to particular embodiments of the instant method, separation of X sperm cells and Y sperm cells can be facilitated by differentiating sperm cells (i) with which AAA associates and/or (ii) to which AAA binds (hence capacitated sperm cells, and expectedly primarily Y sperm cells) from sperm cells that AAA does not associate with and/or bind to (hence non-capacitated sperm cells, and expectedly primarily X sperm cells).

Of course, in addition to the lectins detailed above, it is herein contemplated that other molecules capable of associating with and/or binding to biomarkers which are indicative (i) of at least the initiation of capacitation and/or (ii) that capacitation has been completed may be useful with the present invention as capacitation indicator associators.

As an alternative approach, as to particular embodiments of the instant method, separation of X sperm cells and Y sperm cells can be facilitated by differentiating sperm cells that have not undergone capacitation (expectedly primarily X sperm cells) from sperm cells that have undergone capacitation (expectedly primarily Y sperm cells), for example by selecting for sperm cells comprising a non-capacitated sperm cell indicator which a non-capacitated sperm cell indicator associator associates with, for example via binding to. As a result, the instant method can include employing and/or using a non-capacitated sperm cell indicator which can be associated with non-capacitated sperm cells to differentiate the non-capacitated sperm cells from capacitated sperm cells.

As to particular embodiments, the instant method can include associating a non-capacitated sperm cell indicator associator with a non-capacitated sperm cell indicator to differentiate non-capacitated sperm cells from capacitated sperm cells.

As an illustrative example, the lectin wheat germ agglutinin (WGN), which targets sialic acid (a non-capacitated sperm cell indicator), can be such a non-capacitated sperm cell indicator associator and in accordance with the instant method, can expectedly associate with and/or bind to primarily X sperm cells. Correspondingly, as to particular embodiments of the instant method, separation of X sperm cells and Y sperm cells can be facilitated by differentiating sperm cells (i) with which WGN associates and/or (ii) to which WGN bind (hence non-capacitated sperm cells, and expectedly primarily X sperm cells) from sperm cells that WGN does not associate with and/or bind to (hence capacitated sperm cells, and expectedly primarily Y sperm cells).

As to particular embodiments of the instant method, the capacitation indicator associator or non-capacitated sperm cell indicator associator can be associated with a particle, as detailed below, whereby the particle can be a separable particle such that the particle and its associated capacitation indicator associator or non-capacitated sperm cell indicator associator can be separated from the sample sperm cell population and thereby function to remove the corresponding capacitated sperm cells (expectedly primarily Y sperm cells) or non-capacitated sperm cells (expectedly primarily X sperm cells) from the sample sperm cell population.

### Acrosome Reacted Sperm Cell Differentiation

As to particular embodiments of the instant method, separation of X sperm cells and Y sperm cells can be facilitated by differentiating between sperm cells that have undergone the acrosome reaction or changes that may be associated with the acrosome reaction (hence acrosome-reacted sperm cells) and sperm cells that have not undergone the acrosome reaction (hence non-acrosome-reacted sperm cells), whereby said acrosome reaction can be a cellular process and/or a maturational step which can be an exocytotic event leading to the release of enzymes that aid penetration of the zona pellucida, and to the acquisition of properties by the sperm head plasma membrane that permit fusion with the egg.

As per the present invention, the rate of the acrosome reaction, which is at least in part dependent on the rate of capacitation, can differ between X sperm cells and Y sperm cells, whereby as used herein “rate of the acrosome reaction” can mean the percent of sperm cells that have at least initiated and/or completed the acrosome reaction over time or per unit of time. In particular, as Y sperm cells can undergo capacitation more quickly (or faster) than X sperm cells, Y sperm cells can also undergo the acrosome reaction more quickly (or faster) than X sperm cells, and this difference in acrosome reaction rate can be used to separate X sperm cells and Y sperm cells. Thus, as to particular embodiments of the instant method, separation of X sperm cells and Y sperm cells can be facilitated by differentiating between sperm cells that undergo the acrosome reaction more quickly (expectedly Y sperm cells) and sperm cells that undergo the acrosome reaction more slowly (expectedly X sperm cells).

As per the present invention, the acrosome reaction can result in the gain and/or exposure of a targetable molecule(s) which can subsequently serve as an “acrosome reaction indicator” or a biomarker indicative (i) of at least the initiation of the acrosome reaction and/or (ii) that the acrosome reaction has been completed. Accordingly, acrosome-reacted sperm cells can comprise an acrosome reaction indicator whereas non-acrosome-reacted sperm cells can be void of such an acrosome reaction indicator. As a result, the instant method can include employing and/or using an acrosome reaction indicator which can be associated with acrosome-reacted sperm cells to differentiate the acrosome-reacted sperm cells from non-acrosome-reacted sperm cells.

As to particular embodiments, the instant method can include associating an acrosome reaction indicator associator with an acrosome reaction indicator to differentiate acrosome-reacted sperm cells from non-acrosome-reacted sperm cells.

As but one illustrative example, as per the present invention, in association with the acrosome reaction, equatorin (or MN9 antigen), an acrosomal protein, can translocate to the sperm cell's surface over the equatorial region to participate in fusion; following, equatorin can serve as an acrosome reaction indicator. Equatorin can be recognized by an equatorin antibody (or EQTN antibody or MN9 antibody) and thus, said equatorin antibody can serve as an acrosome reaction indicator associator.

Accordingly, as to particular embodiments of the instant method, separation of X sperm cells and Y sperm cells can be facilitated by differentiating sperm cells that have undergone the acrosome reaction (expectedly primarily Y sperm cells) from sperm cells that have not undergone the acrosome reaction (expectedly primarily X sperm cells) by selecting for sperm cells comprising an acrosome reaction indicator which an acrosome reaction indicator associator associates with, for example via binding to.

Thus, as to particular embodiments of the instant method, separation of X sperm cells and Y sperm cells can be facilitated by differentiating sperm cells (i) with which an equatorin antibody associates and/or (ii) to which an equatorin antibody binds (hence acrosome-reacted sperm cells, and expectedly primarily Y sperm cells) from sperm cells that the equatorin antibody does not associate with and/or bind to (hence non-acrosome-reacted sperm cells, and expectedly primarily X sperm cells).

Of course, in addition to the equatorin antibody detailed above, it is herein contemplated that other molecules capable of associating with and/or binding to biomarkers which are indicative (i) of at least the initiation of the acrosome reaction and/or (ii) that the acrosome reaction has been completed may be useful with the present invention as acrosome reaction indicator associators.

Of note, in addition to differentiating and separating Y sperm cells that have undergone the acrosome reaction from the sample sperm cell population, the instant method may also be useful for differentiating X sperm cells that have undergone the acrosome reaction, thus facilitating separation of these sperm cells from the sample sperm cell population. Such a separation may be beneficial, as acrosome-reacted sperm cells may be non-viable and/or unable to fertilize an egg to produce offspring and correspondingly, can be removed from the desired subpopulation via the instant method.

As to particular embodiments of the instant method, the acrosome reaction indicator associator can be associated with a particle, as detailed below, whereby the particle can be a separable particle such that the particle and its associated acrosome reaction indicator associator can be separated from the sample sperm cell population and thereby function to remove the corresponding acrosome-reacted sperm cells (expectedly primarily Y sperm cells) from the sample sperm cell population.

### Separable Particles

As stated above, the instant method can involve a particle with which a capacitation indicator associator, a non-capacitated sperm cell indicator associator, and/or an acrosome reaction indicator associator can associate with, whereby the particle can be a separable particle meaning the particle can be capable of being separated from the sample sperm cell population.

As to particular embodiments, the particle can be separated from the sample sperm cell population by the application of a force to which the particle can be responsive. For example, the particle can be responsive to a centrifugal force, an electrostatic force, a gravitational force, a magnetic force, or the like, or combinations thereof; thus, the instant method can include applying such a force to the sample sperm cell population following the induction of capacitation.

As but one illustrative example, the instant method can employ a particle which can be responsive to a magnetic force and correspondingly, the particle can be magnetic, meaning exhibiting magnetic properties in the presence of an external magnetic field. Following, the particle can be a magnetic particle.

As to particular embodiments, the particle can be a magnetic nanoparticle.

As to particular embodiments, the particle can be a superparamagnetic iron oxide nanoparticle (SPION).

As to particular embodiments, the particle can be a hydrophobic SPION, which can mean covered with hydrophobic moieties.

As to particular embodiments, the hydrophobic SPION can be encapsulated within a block copolymer (BCP), which may comprise covalently linked hydrophobic and hydrophilic polymers, thus forming water-soluble colloidally stable polymer nanocomposites. As to particular embodiments, polymer nanocomposite synthesis can be facilitated by electrohydrodynamic mixing-mediated nanoprecipitation, which can involve rapid mixing induced by electrohydrodynamics, whereby such a synthesis can (i) effectively control the size of the polymer nanocomposites, and (ii) produce a narrow size distribution of the polymer nanocomposites.

As to particular embodiments, the BCP can comprise polyethylene glycol (PEG) as a hydrophilic polymer and polystyrene (PS) as a hydrophobic polymer.

As but one illustrative example, the hydrophobic SPION can have a particle size of about 15 nm, the PEG can be PEG 20,000 (having a molecular weight of about 20,000 g/mol), and the PS can be PS 9,500 (having a molecular weight of about 9,500 g/mol). For synthesis, the electrospray can be performed at about −1,000 V, a SPION iron oxide:BCP ratio of about 1:2 can be used (for example about 1.5 mg iron oxide to about 3 mg BCP), and an organic phase:aqueous phase ratio of about 1:20 can be used (for example about 0.5 mL organic phase to about 10 mL aqueous phase, such as water). Following synthesis, the magnetic nanoparticles can be recovered via a centrifugal filter, whereby said filter can have a 100 kDa molecular weight cut-off (MWCO). Additionally, the magnetic nanoparticles can be recovered via magnetic separation, for example with a column having a column matrix composed of ferromagnetic spheres. After recovery, the magnetic nanoparticles can be characterized for properties such as hydrodynamic size, charge, and iron concentration. As to particular embodiments, the resultant magnetic nanoparticles can have a mean size of about 185 nanometers.

Additional magnetic nanoparticles which may be useful with the present invention can include those particles disclosed in International Publication No. WO 2019/094831 and United States Patent Application Publication No. 2021/0230541, each of which is hereby incorporated by reference herein in its entirety.

For use with particular embodiments of the instant method which include magnetically separating X sperm cells and Y sperm cells by differentiating sperm cells that have undergone capacitation (expectedly primarily Y sperm cells) from sperm cells that have not undergone capacitation (expectedly primarily X sperm cells), a capacitation indicator associator can be associated with a magnetic particle (such as via conjugation) to provide a capacitation indicator associator-magnetic particle.

As an illustrative example, a lectin, such as PNA, can be conjugated to a magnetic particle, whereby both PNA and the magnetic particle can be activated for said conjugation. Regarding the former, PNA can be activated via a reaction with 6-Azidohexanoic acid sulfo-NHS ester to provide activated PNA. The magnetic particle can be activated via a reaction with DBCO-PEG NHS ester to provide an activated magnetic particle. Following, the activated PNA can be combined with the activated magnetic particle and a conjugation reaction can occur to provide PNA-magnetic particles. As to particular embodiments, the resultant PNA-magnetic particles can comprise (i) an iron oxide concentration of about 0.2 mg/mL, (ii) a PNA concentration of about 140 ng PNA/μg of particle, (iii) a particle concentration of about 4.6×10¹⁰ particles/mL, (iv) a size of about 154 nm, and (v) a zeta potential (in 1 mM KCl at pH 7.05) of about −3 mV.

For use with particular embodiments of the instant method which include separating X sperm cells and Y sperm cells by differentiating sperm cells that have undergone the acrosome reaction (expectedly primarily Y sperm cells) from sperm cells that have not undergone the acrosome reaction (expectedly primarily X sperm cells), an acrosome reaction indicator associator can be associated with a magnetic particle (such as via conjugation) to provide an acrosome reaction indicator associator-magnetic particle.

As an illustrative example, an equatorin antibody can be conjugated to a magnetic particle, whereby both the equatorin antibody and the magnetic particle can be activated for said conjugation. Concerning the equatorin antibody, prior to activation, the thiol groups can be reduced, for example via mercaptoethylamine (MEA). Subsequently, the equatorin antibody can be activated via a reaction with TCO-sulfo maleimide to provide activated equatorin antibody. The magnetic particle can be activated via a reaction with TZ-PEG NHS ester to provide an activated magnetic particle. Following, the activated equatorin antibody can be combined with the activated magnetic particle and a conjugation reaction can occur to provide equatorin antibody-magnetic particles. As to particular embodiments, the resultant equatorin antibody-magnetic particles can comprise (i) an iron oxide concentration of about 0.5 mg/mL, (ii) an equatorin antibody concentration of about 25 ng antibody/μL, (iii) a particle concentration of about 1.1×10″ particles/mL, (iv) a size of about 167 nm, and (v) a zeta potential (in 1 mM KCl at pH 7.05) of about −0.3 mV.

As to particular embodiments, a capacitation indicator associator-magnetic particle or an acrosome reaction indicator associator-magnetic particle can have a size of: less than about 1,000 nm, less than about 900 nm, less than about 800 nm, less than about 700 nm, less than about 600 nm, less than about 500 nm, less than about 400 nm, less than about 300 nm, less than about 200 nm, or less than about 100 nm.

### Separation of Capacitated Sperm Cells

After the sample sperm cell population has been exposed to a capacitation inducer for a period of time sufficient to induce capacitation of at least a portion of the Y sperm cells within the sample sperm cell population to generate a capacitation-induced sperm cell subpopulation, the sperm cells that have undergone capacitation can be separated from the sample sperm cell population, whereby this separation process can have very little or essentially no effect(s) on the non-capacitated X sperm cells, and specifically can have very little or essentially no negative effect(s) on the non-capacitated X sperm cells, and more specifically, can have very little or essentially no negative effect(s) on the non-capacitated X sperm cells' ability to subsequently fertilize an egg to produce offspring.

Now referring primarily to FIG. 3, as to particular embodiments, the instant method can include exposing the capacitation-induced sperm cell subpopulation to capacitation indicator associator-magnetic particles, for example by combining the sample sperm cell population (and correspondingly the capacitation-induced sperm cell subpopulation) and the capacitation indicator associator-magnetic particles (preferably in a protein-free buffer). During exposure, the capacitation indicator associator can associate with and/or bind to the capacitated sperm cells (expectedly primarily Y sperm cells) via a capacitation indicator to effectively associate the capacitated sperm cells with the magnetic particles to generate capacitated sperm cell-magnetic particle complexes.

In detail, the capacitation-induced sperm cell subpopulation can be exposed to the capacitation indicator associator-magnetic particles for a period of time at a selected temperature, whereby said period of time can be sufficient to induce association of at least a portion of the capacitated sperm cells (expectedly primarily Y sperm cells) with the magnetic particles. To facilitate said association, the combined capacitation-induced sperm cell subpopulation and capacitation indicator associator-magnetic particles can be rocked on a rocker, for example at room temperature. As but one illustrative example, the period of time can be about ten (10) minutes. As but one illustrative example, the selected temperature can be in a range of between about 20° Celsius to about 40° Celsius.

Following, the capacitated sperm cell-magnetic particle complexes can be separated from the sample sperm cell population in the presence of a magnetic field such as can be generated by a magnet which attracts the magnetic particles and correspondingly the associated capacitated sperm cells, hence the capacitated sperm cell-magnetic particle complexes.

Concerning the magnet, embodiments of the described methods and processes may be used with any type of magnetically identifying separating apparatus, including but not limited to devices incorporating columns, such as magnetic-activated cell sorting (MACS) products, devices using simple magnetic fields applied to test tubes or containers, or high throughput magnetic devices.

As but one illustrative example, a 1.3 Tesla magnet may be useful for attracting the capacitated sperm cell-magnetic particle complexes. As but a second one illustrative example, a 3 Tesla magnet may be useful for attracting the capacitated sperm cell-magnetic particle complexes. As but a third illustrative example, a 1.4 Tesla Halbach array magnet may be useful for attracting the capacitated sperm cell-magnetic particle complexes.

Concerning methodology, the capacitated sperm cell-magnetic particle complexes can be exposed to the magnet for a period of time at a selected temperature, whereby said period of time can be sufficient to permit attraction of the capacitated sperm cell-magnetic particle complexes to the magnet and corresponding movement or migration of the capacitated sperm cell-magnetic particle complexes through the fluid medium toward and/or to the magnet. As but one illustrative example, the period of time can be about twenty (20) minutes. As but one illustrative example, the selected temperature can be in a range of between about 20° Celsius to about 40° Celsius.

As to particular embodiments of the instant method, it can be that particles having a greater magnetic composition, for example a greater iron content, can move toward and/or to the magnet with a greater velocity, which may result in a decrease in the period of time needed for exposure to the magnet.

After separation of the capacitated sperm cell-magnetic particle complexes from the sample sperm cell population via the magnet, at least one of the capacitated sperm cells (associated with the magnet, expectedly primarily Y sperm cells) or the non-capacitated sperm cells (not associated with the magnet, expectedly primarily X sperm cells) can be collected.

As an illustrative example, the non-capacitated sperm cells (expectedly primarily X sperm cells) can be collected via aspiration.

As to particular embodiments, the non-capacitated sperm cells (expectedly primarily X sperm cells) can be the desired subpopulation of sperm cells.

### Separation of Acrosome-Reacted Sperm Cells

After the sample sperm cell population has been exposed to a capacitation inducer for a period of time sufficient to induce capacitation of at least a portion of the Y sperm cells within the sample sperm cell population to generate a capacitation-induced sperm cell subpopulation, the sperm cells that have undergone the acrosome reaction can be separated from the sample sperm cell population, whereby this separation process can have very little or essentially no effect(s) on the non-acrosome-reacted X sperm cells, and specifically can have very little or essentially no negative effect(s) on the non-acrosome-reacted X sperm cells, and more specifically, can have very little or essentially no negative effect(s) on the non-acrosome-reacted X sperm cells' ability to subsequently fertilize an egg to produce offspring.

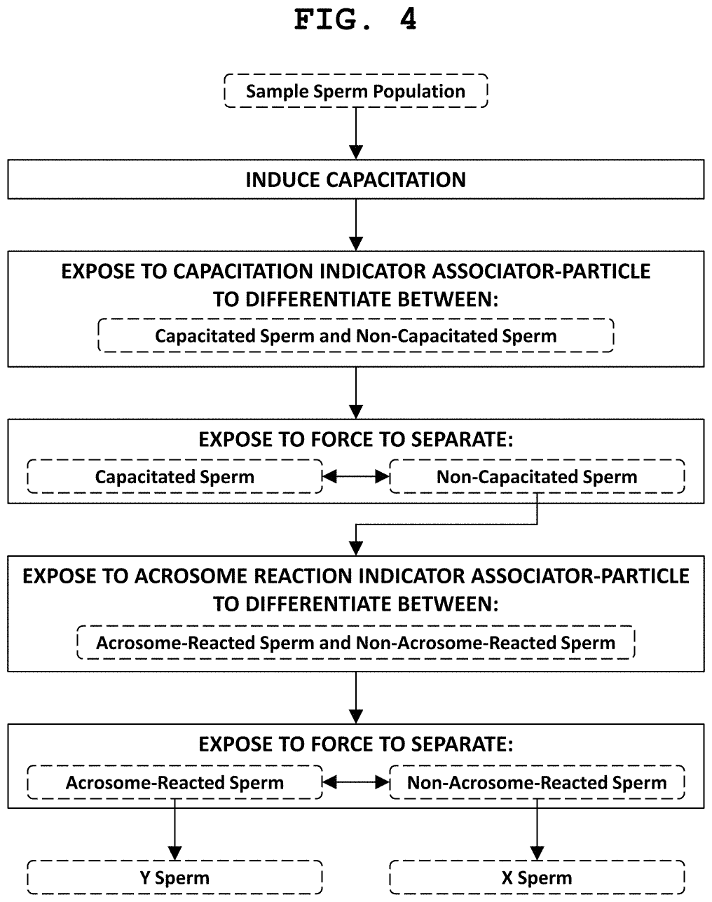

Now referring primarily to FIG. 4, as to particular embodiments, the instant method can include exposing the capacitation-induced sperm cell subpopulation to acrosome reaction indicator associator-magnetic particles, for example by combining the sample sperm cell population (and correspondingly the capacitation-induced sperm cell subpopulation) and the acrosome reaction indicator associator-magnetic particles (preferably in a protein-free buffer). During exposure, the acrosome reaction indicator associator can associate with and/or bind to the acrosome-reacted sperm cells (expectedly primarily Y sperm cells) via an acrosome reaction indicator to effectively associate the acrosome-reacted sperm cells with the magnetic particles to generate acrosome-reacted sperm cell-magnetic particle complexes.

In detail, the capacitation-induced sperm cell subpopulation can be exposed to the acrosome reaction indicator associator-magnetic particles for a period of time at a selected temperature, whereby said period of time can be sufficient to induce association of at least a portion of the acrosome-reacted sperm cells (expectedly primarily Y sperm cells) with the magnetic particles. To facilitate said association, the combined capacitation-induced sperm cell subpopulation and acrosome reaction indicator associator-magnetic particles can be rocked on a rocker, for example at room temperature. As but one illustrative example, the period of time can be about ten (10) minutes. As but one illustrative example, the selected temperature can be in a range of between about 20° Celsius to about 40° Celsius.

Following, the acrosome-reacted sperm cell-magnetic particle complexes can be separated from the sample sperm cell population in the presence of a magnetic field such as can be generated by a magnet which attracts the magnetic particles and correspondingly the associated acrosome-reacted sperm cells, hence the acrosome-reacted sperm cell-magnetic particle complexes.

Concerning the magnet, embodiments of the described methods and processes may be used with any type of magnetically identifying separating apparatus, including but not limited to devices incorporating columns, such as magnetic-activated cell sorting (MACS) products, devices using simple magnetic fields applied to test tubes or containers, or high throughput magnetic devices.

As but one illustrative example, a 1.3 Tesla magnet may be useful for attracting the acrosome-reacted sperm cell-magnetic particle complexes. As but a second one illustrative example, a 3 Tesla magnet may be useful for attracting the acrosome-reacted sperm cell-magnetic particle complexes. As but a third illustrative example, a 1.4 Tesla Halbach array magnet may be useful for attracting the acrosome-reacted sperm cell-magnetic particle complexes.

Concerning methodology, the acrosome-reacted sperm cell-magnetic particle complexes can be exposed to the magnet for a period of time at a selected temperature, whereby said period of time can be sufficient to permit attraction of the acrosome-reacted sperm cell-magnetic particle complexes to the magnet and corresponding movement or migration of the acrosome-reacted sperm cell-magnetic particle complexes through the fluid medium toward and/or to the magnet. As but one illustrative example, the period of time can be about twenty (20) minutes. As but one illustrative example, the selected temperature can be in a range of between about 20° Celsius to about 40° Celsius.

As to particular embodiments of the instant method, it can be that particles having a greater magnetic composition, for example a greater iron content, can move toward and/or to the magnet with a greater velocity, which may result in a decrease in the period of time needed for exposure to the magnet.

After separation of the acrosome-reacted sperm cell-magnetic particle complexes from the sample sperm cell population via the magnet, at least one of the acrosome-reacted sperm cells (associated with the magnet, expectedly primarily Y sperm cells) or the non-acrosome-reacted sperm cells (not associated with the magnet, expectedly primarily X sperm cells) can be collected.

As an illustrative example, the non-acrosome-reacted sperm cells (expectedly primarily X sperm cells) can be collected via aspiration.

As to particular embodiments, the non-acrosome-reacted sperm cells (expectedly primarily X sperm cells) can be the desired subpopulation of sperm cells.

### Separation of Dead Sperm Cells

As stated above, as to particular embodiments, the instant method can further include (i) differentiating between sperm cells that have undergone a cellular process and sperm cells that have not undergone the cellular process, and (ii) separating the sperm cells that have undergone the cellular process and the sperm cells that have not undergone the cellular process.

As to particular embodiments of the instant method, the cellular process can be cell death. Correspondingly, sperm cells can be either viable sperm cells (which, of course, have not undergone cell death) or non-viable sperm cells (which can include dying or dead sperm cells). Typically, viable sperm cells can have an intact plasma membrane whereas the plasma membrane of non-viable sperm cells can be compromised or damaged.

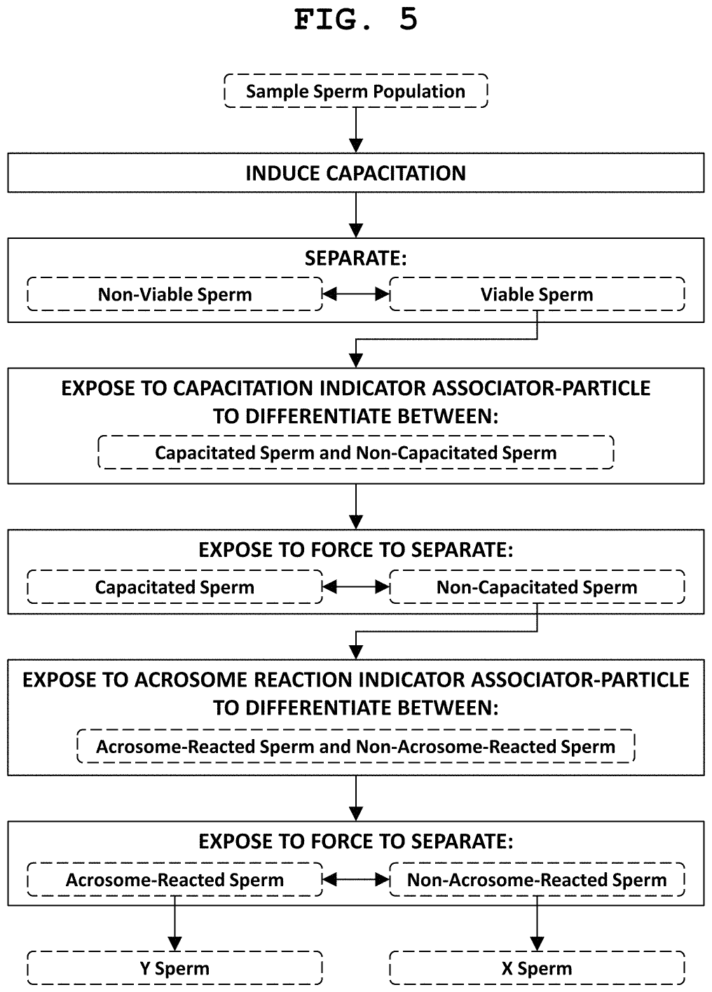

Following, particular embodiments of the instant method can include (i) differentiating between non-viable sperm cells and viable sperm cells, and (ii) separating the non-viable sperm cells and the viable sperm cells, as shown in FIG. 5.

As to particular embodiments of the instant method, non-viable sperm cells and viable sperm cells can be separated by a swim-up separation method, a density gradient separation method (such as a Percoll separation method), a separation method utilizing a negatively charged material (such as glass, silica, dextran, metal oxides, etc.), or any known method for separating non-viable sperm cells and viable sperm cells.

As to particular embodiments of the instant method, particles having an electrical charge or zeta potential can be used to separate non-viable sperm cells and viable sperm cells.

As to particular embodiments of the instant method, particles having a net negative electrical charge or zeta potential can be used to separate non-viable sperm cells and viable sperm cells, as negatively charged particles may bind specifically to compromised, damaged, dying, or dead sperm cells via an electrical charge interaction (as disclosed in U.S. Pat. No. 10,324,086, which is hereby incorporated by reference herein in its entirety).

As to particular embodiments, the negative charge of the particles can be facilitated by a chargeable compound, such as one which may coat at least a portion of the particle.

As to particular embodiments, the particles can be functionalized by an amino group which can provide and/or contribute to the negative charge.

As to particular embodiments, the particles can be functionalized by a carboxyl group which can provide and/or contribute to the negative charge.

As to particular embodiments and with reference to U.S. Pat. No. 10,324,086, the particles can include a chargeable silicon-containing compound which may coat at least a portion of the particle. As to particular embodiments, carboxyl group functionalized silane coated particles may be used (such as without further surface manipulation) since the carboxyl group on the silane can contribute to the particles having a net negative electrical charge or zeta potential.

As to particular embodiments, the particles can be magnetic particles.

As to particular embodiments, the particles can be magnetic nanoparticles, such as the illustrative exemplary magnetic nanoparticles taught in U.S. Pat. No. 10,324,086.

### Particles and Kit

As to particle embodiments, capacitation indicator associator-magnetic particles can be provided.

As to particular embodiments, acrosome reaction indicator associator-magnetic particles can be provided.

As to particular embodiments, means for separating non-viable sperm cells and viable sperm cells can be provided.

As to particular embodiments, a kit comprising one or more of (i) capacitation indicator associator-magnetic particles, (ii) acrosome reaction indicator associator-magnetic particles, or (iii) means for separating non-viable sperm cells and viable sperm cells can be provided, whereby the kit may be useful for facilitating the instant method.

### Order of Separations

The instant method can include (i) separation of capacitated sperm cells, namely separating capacitated sperm cells (expectedly primarily Y sperm cells) and non-capacitated sperm cells (expectedly primarily X sperm cells), (ii) separation of acrosome-reacted sperm cells, namely separating acrosome-reacted sperm cells (expectedly primarily Y sperm cells) and non-acrosome-reacted sperm cells (expectedly primarily X sperm cells), and (iii) separation of non-viable sperm cells, namely separating non-viable sperm cells and viable sperm cells.

As to particular embodiments, the instant method can include simultaneous (or concurrent) separation of capacitated sperm cells, acrosome-reacted sperm cells, and non-viable sperm cells.

As to other particular embodiments, the instant method can include stepwise separation of capacitated sperm cells, acrosome-reacted sperm cells, and non-viable sperm cells.

As to particular embodiments including stepwise separation, the instant method can include two discrete separation steps.

As one illustrative example, a first separation step can include separation of non-viable sperm cells, and a second separation step can include simultaneous separation of capacitated sperm cells and acrosome-reacted sperm cells.

As a second illustrative example, a first separation step can include simultaneous separation of capacitated sperm cells and acrosome-reacted sperm cells, and a second separation step can include separation of non-viable sperm cells.

As a third illustrative example, a first separation step can include separation of capacitated sperm cells, and a second separation step can include simultaneous separation of acrosome-reacted sperm cells and non-viable sperm cells.

As a fourth illustrative example, a first separation step can include simultaneous separation of acrosome-reacted sperm cells and non-viable sperm cells, and a second separation step can include separation of capacitated sperm cells.

As a fifth illustrative example, a first separation step can include separation of acrosome-reacted sperm cells, and a second separation step can include simultaneous separation of non-viable sperm cells and capacitated sperm cells.

As a sixth illustrative example, a first separation step can include simultaneous separation of non-viable sperm cells and capacitated sperm cells, and a second separation step can include separation of acrosome-reacted sperm cells.

As to particular embodiments including stepwise separation, the instant method can include three discrete separation steps.

As one illustrative example, a first separation step can include separation of non-viable sperm cells, a second separation step can include separation of capacitated sperm cells, and a third separation step can include separation of acrosome-reacted sperm cells.

As a second illustrative example, a first separation step can include separation of non-viable sperm cells, a second separation step can include separation of acrosome-reacted sperm cells, and a third separation step can include separation of capacitated sperm cells.

As a third illustrative example, a first separation step can include separation of capacitated sperm cells, a second separation step can include separation of acrosome-reacted sperm cells, and a third separation step can include separation of non-viable sperm cells.

As a fourth illustrative example, a first separation step can include separation of capacitated sperm cells, a second separation step can include separation of non-viable sperm cells, and a third separation step can include separation of acrosome-reacted sperm cells.

As a fifth illustrative example, a first separation step can include separation of acrosome-reacted sperm cells, a second separation step can include separation of non-viable sperm cells, and a third separation step can include separation of capacitated sperm cells.

As a sixth illustrative example, a first separation step can include separation of acrosome-reacted sperm cells, a second separation step can include separation of capacitated sperm cells, and a third separation step can include separation of non-viable sperm cells.

Following separation, the desired subpopulation of sperm cells can be collected.

As to particular embodiments, the desired subpopulation of sperm cells can comprise primarily X sperm cells.

As to particular embodiments, the desired subpopulation of sperm cells can comprise primarily Y sperm cells.

After collection, the desired subpopulation of sperm cells can be used for many applications, including but not limited to fertilization processes such as artificial insemination (AI), in vitro fertilization (IVF), etc.

### Sample Sperm Cell Population Characterization

As to particular embodiments, the instant method can further include characterizing the sample sperm cell population at one or more points during the separation process, whereby such a characterization can provide at least an estimate of the number of sperm cells having the assessed characteristic. Correspondingly, the method component(s) and amount(s) thereof needed to facilitate separation of the sperm cells having the assessed characteristic can be determined.

As a first example, the number of non-viable sperm cells within the sample sperm cell population can be determined. Correspondingly, the method component(s) and amount(s) thereof needed to separate the non-viable sperm cells and viable sperm cells can be determined. Concerning methodology, as but one illustrative example, the number of non-viable sperm cells can be determined via propidium iodide (PI) staining, such as via flow cytometry.

As a second example, the number of capacitated sperm cells within the sample sperm cell population can be determined. Correspondingly, the method component(s) and amount(s) thereof needed to separate the capacitated sperm cells and non-capacitated sperm cells can be determined. Concerning methodology, as but one illustrative example, the number of capacitated sperm cells can be determined via a capacitation indicator (for example PNA), such as via flow cytometry.

As a third example, the number of acrosome-reacted sperm cells within the sample sperm cell population can be determined. Correspondingly, the method component(s) and amount(s) thereof needed to separate the acrosome-reacted sperm cells and non-acrosome-reacted sperm cells can be determined. Concerning methodology, as but one illustrative example, the number of acrosome-reacted sperm cells can be determined via an acrosome reaction indicator (for example an MN9 antibody), such as via flow cytometry.

As to particular embodiments, to further determine the method component(s) and amount(s) thereof needed for separation, the diameter and/or surface area of the sperm cell's head can be taken into account; following, knowing the diameter and/or cross-section area and/or surface area of the particle and the packing density of the particles, the number of particles needed for separation can be determined.

### Sample Sperm Cell Population

The instant method may be useful for separating X sperm cells and Y sperm cells within a sample sperm cell population, whereby the sample can comprise human sperm cells or non-human sperm cells. Regarding the latter, the instant method may be useful for separating livestock sperm cells, such as bovine sperm cells, equine sperm cells, porcine sperm cells, caprine sperm cells, ovine sperm cells, etc. Also regarding the latter, the instant method may be useful for separating canine sperm cells, feline sperm cells, etc.

The instant method may be useful for separating X sperm cells and Y sperm cells within a sample sperm cell population, whereby the method may be applied to sperm cells contained in freshly collected neat ejaculates, after dilution, during and after cooling, or during and after other semen processing procedures that may be employed prior to cryopreservation, or to frozen-thawed sperm cells.

### Conceivable Advantages

As to particular embodiments, the instant method may provide a faster separation process than prior methods of sperm sorting.

As to particular embodiments, the instant method may provide an environment having less potential for damaging the desired subpopulation of sperm cells than prior methods of sperm sorting.

As to particular embodiments, the instant method may provide a greater purity of the desired subpopulation of sperm cells than prior methods of sperm sorting.

As to particular embodiments, the instant method may provide a greater yield of the desired subpopulation of sperm cells than prior methods of sperm sorting.

As to particular embodiments, the instant method may provide a less expensive separation process than prior methods of sperm sorting.

As to particular embodiments, the instant method may provide an easier separation process than prior methods of sperm sorting.

As to particular embodiments, the instant method may provide a separation process which requires less equipment than prior methods of sperm sorting.

As to particular embodiments, the instant method may provide a separation process which requires less complex equipment than prior methods of sperm sorting.

As to particular embodiments, the instant method may provide a separation process which requires less technical expertise than prior methods of sperm sorting.

As to particular embodiments, the instant method may provide a separation process which can be conducted in the field, as opposed to in a laboratory or laboratory-like setting.

### Example

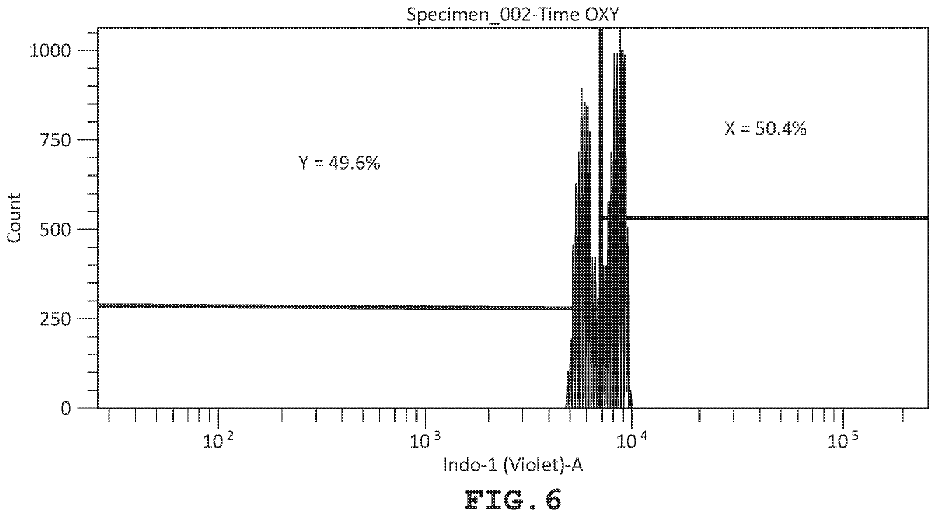

A fresh ejaculate was collected, and the baseline pHₑ was determined to be 5.93. The ejaculate was spun at 1,800 RPMs for 15 minutes; following, the seminal plasma was aspirated and discarded. The sperm cell pellet was resuspended in sperm TALP buffer (BGM-1, pH 7.3). As assessed via Hoechst staining/flow cytometry, at Time 0, the sample contained 49.6% Y sperm cells and 50.4% X sperm cells (as shown in FIG. 6).

The sperm cells were then aliquoted into three samples, each comprising 10 million sperm cells, whereby Sample A was incubated for 3 hours, Sample B was incubated for 5 hours, and Sample C was incubated for 24 hours. All incubations were done in an incubator at about 37° C. in about 5% CO₂.

Following incubation, each sample was characterized for its number of non-viable sperm cells (via PI staining/flow cytometry); Sample A contained about 28.8% non-viable sperm cells (equating to about 2.8 million sperm cells), Sample B contained about 20.5% non-viable sperm cells (equating to about 2 million sperm cells), and Sample C contained about 71.7% non-viable sperm cells (equating to about 7.2 million sperm cells).

For separation of the non-viable sperm cells, the number of negatively charged magnetic nanoparticles needed per sperm cell was determined based on the sperm cell surface area, the negatively charged magnetic nanoparticle surface area, and a hexagonal packing density, whereby such a calculation indicated that about 4.9 billion negatively charged magnetic nanoparticles, 3.4 billion negatively charged magnetic nanoparticles, and 12.2 billion negatively charged magnetic nanoparticles should be used for Sample A, Sample B, and Sample C, respectively. After the particles were added to the sperm cells, the combination was incubated on a rocker for 10 minutes at room temperature, and then magnetically collected via exposure to a 1.4 Tesla Halbach array magnet for 20 minutes.

The nonmagnetic sperm cells were aspirated and then each sample was characterized for its number of capacitated sperm cells (via PNA-FITC staining/flow cytometry); Sample A contained about 33.4% capacitated sperm cells (equating to about 2.3 million sperm cells), Sample B contained about 20% capacitated sperm cells (equating to about 1.6 million sperm cells), and Sample C contained about 76.4% capacitated sperm cells (equating to about 2.1 million sperm cells).

For separation of the capacitated sperm cells, the number of PNA-magnetic nanoparticles needed per sperm cell was determined, based on the sperm cell surface area, the PNA-magnetic nanoparticle surface area, and a hexagonal packing density, whereby such a calculation indicated that about 3.8 billion PNA-magnetic nanoparticles, 2.7 billion PNA-magnetic nanoparticles, and 3.6 billion PNA-magnetic nanoparticles should be used for Sample A, Sample B, and Sample C, respectively. After the particles were added to the sperm cells, the combination was incubated on a rocker for 10 minutes at room temperature, and then magnetically collected via exposure to a 1.4 Tesla Halbach array magnet for 20 minutes.

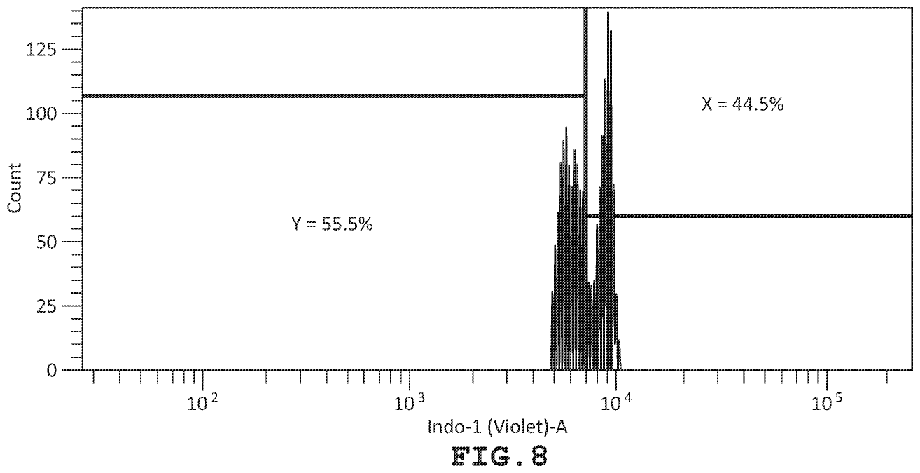

Regarding Sample B, the nonmagnetic sperm cells were aspirated and then the X sperm cell and Y sperm cell content was determined via Hoechst staining/flow cytometry (as shown in FIG. 8).

Regarding Samples A and C, the nonmagnetic sperm cells were aspirated and then each sample was characterized for its number of acrosome-reacted sperm cells (via EQTN antibody-Alexa staining/flow cytometry); Sample A contained about 4% acrosome-reacted sperm cells (equating to about 284 thousand sperm cells), and Sample C contained about 28.6% acrosome-reacted sperm cells (equating to about 200 thousand sperm cells).

For separation of the acrosome-reacted sperm cells, the number of EQTN antibody-magnetic nanoparticles needed per sperm cell was determined, based on the sperm cell surface area, the EQTN antibody-magnetic nanoparticle surface area, and a hexagonal packing density, whereby such a calculation indicated that about 482 million EQTN antibody-magnetic nanoparticles, and 321 million EQTN antibody-magnetic nanoparticles should be used for Sample A and Sample C, respectively. After the particles were added to the sperm cells, the combination was incubated on a rocker for 10 minutes at room temperature, and then magnetically collected via exposure to a 1.4 Tesla Halbach array magnet for 20 minutes.

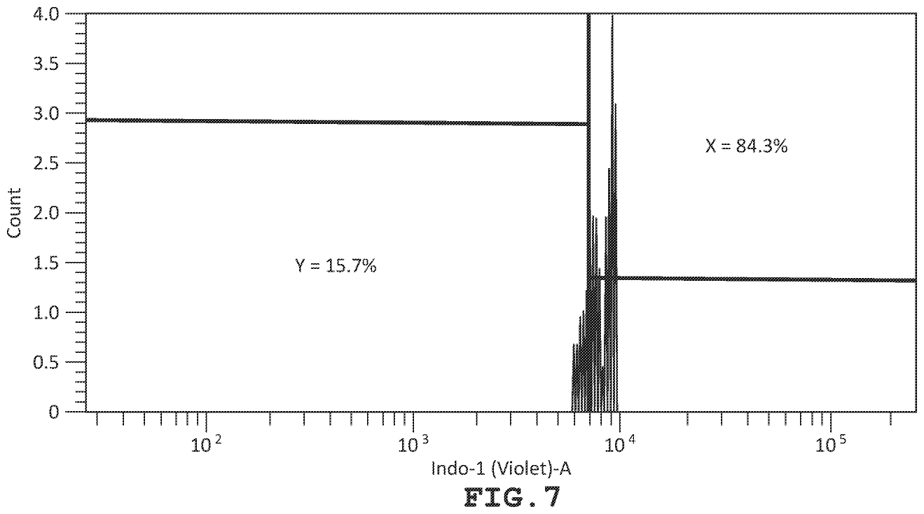

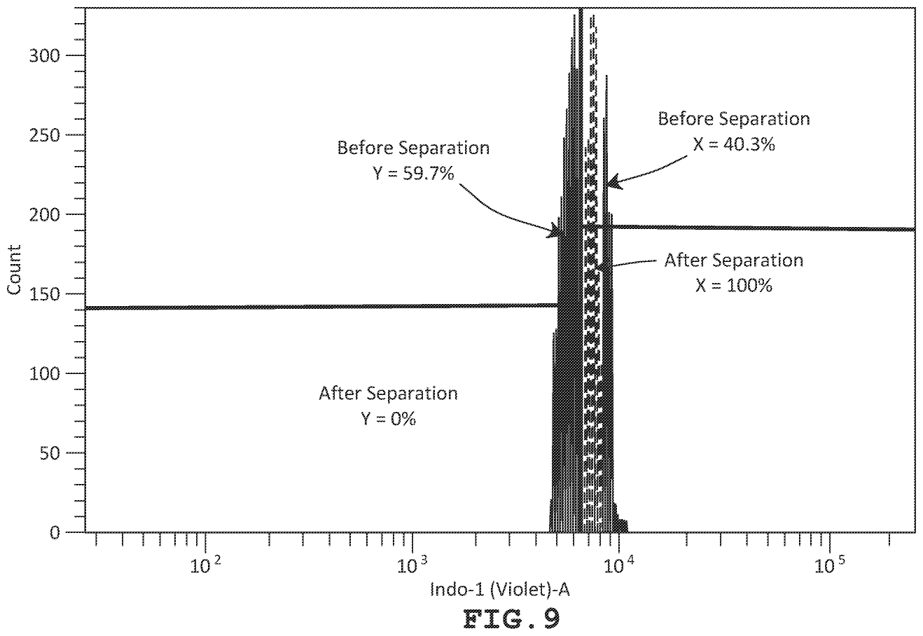

The nonmagnetic sperm cells were aspirated and then the X sperm cell and Y sperm cell content was determined via Hoechst staining/flow cytometry (as shown in FIG. 7 for Sample A and FIG. 9 for Sample C).

As per the results, following the 3 hour incubation (Sample A) and magnetic separation of (i) non-viable sperm cells, (ii) capacitated sperm cells, and (iii) acrosome-reacted sperm cells, the final sperm cell population (theoretically comprising about 4.5 million sperm cells) contained 84.3% X sperm cells and 15.7% Y sperm cells. Following the 24 hour incubation (Sample C) and magnetic separation of (i) non-viable sperm cells, (ii) capacitated sperm cells, and (iii) acrosome-reacted sperm cells, the final sperm cell population (theoretically comprising about 500 thousand sperm cells) contained 100% X sperm cells and 0% Y sperm cell. Notably, following the 5 hour incubation (Sample B) and magnetic separation of only (i) non-viable sperm cells and (ii) capacitated sperm cells, the final sperm cell population (theoretically comprising about 6.4 million sperm cells) contained 44.5% X sperm cells and 55.5% Y sperm cells, suggesting that separation/removal of (i) non-viable sperm cells, (ii) capacitated sperm cells, and (iii) acrosome-reacted sperm cells yields a greater purity of X sperm cells in the final sperm cell population relative to separation/removal of only (i) non-viable sperm cells and (ii) capacitated sperm cells.

As can be easily understood from the foregoing, the basic concepts of the present invention may be embodied in a variety of ways. The invention involves numerous and varied embodiments of a method for separating X chromosome-bearing sperm cells and Y chromosome-bearing sperm cells, including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “associator” should be understood to encompass disclosure of the act of “associating”—whether explicitly discussed or not—and, conversely, were there effectively disclosure of the act of “associating”, such a disclosure should be understood to encompass disclosure of an “associator” and even a “means for associating”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a wearer of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the methods for separating X chromosome-bearing sperm cells and Y chromosome-bearing sperm cells herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A method for separating X chromosome-bearing sperm cells and Y chromosome-bearing sperm cells within a sample sperm cell population, comprising:
differentiating between sperm cells that have undergone a cellular process and sperm cells that have not undergone said cellular process; and
separating said sperm cells that have undergone said cellular process and said sperm cells that have not undergone said cellular process;
wherein a majority of said sperm cells that have undergone said cellular process comprise one of said X chromosome-bearing sperm cells or said Y chromosome-bearing sperm cells; and
wherein a majority of said sperm cells that have not undergone said cellular process comprise the other of said X chromosome-bearing sperm cells or said Y chromosome-bearing sperm cells.

2. (canceled)

3. The method of claim 1, further comprising:
differentiating between sperm cells that have undergone a maturational step and sperm cells that have not undergone said maturational step; and
separating said sperm cells that have undergone said maturational step and said sperm cells that have not undergone said maturational step;
wherein a majority of said sperm cells that have undergone said maturational step comprise one of said X chromosome-bearing sperm cells or said Y chromosome-bearing sperm cells; and
wherein a majority of said sperm cells that have not undergone said maturational step comprise the other of said X chromosome-bearing sperm cells or said Y chromosome-bearing sperm cells.

4. The method of claim 3, wherein said maturational step comprises capacitation; and
wherein said differentiating between sperm cells that have undergone said capacitation and sperm cells that have not undergone said capacitation is facilitated by Y chromosome-bearing sperm cells undergoing said capacitation more quickly than X chromosome-bearing sperm cells.

5-6. (canceled)

7. The method of claim 4, further comprising inducing said capacitation to generate a capacitation-induced sperm cell subpopulation in which said capacitation is induced in at least a portion of said Y chromosome-bearing sperm cells.

8-20. (canceled)

21. The method of claim 7, further comprising employing a capacitation indicator which is associated with said capacitated sperm cells to differentiate said capacitated sperm cells from said non-capacitated sperm cells.

22. The method of claim 21, furthering comprising associating a capacitation indicator associator with said capacitation indicator to differentiate said capacitated sperm cells from said non-capacitated sperm cells.

23. The method of claim 22, wherein said capacitation indicator comprises a saccharide.

24. (canceled)

25. The method of claim 23, wherein said capacitation indicator associator comprises a lectin which associates with said saccharide.

26-39. (canceled)

40. The method of claim 22, further comprising differentiating between sperm cells that have undergone the acrosome reaction and sperm cells that have not undergone said acrosome reaction;
wherein said sperm cells that have undergone said acrosome reaction comprise acrosome-reacted sperm cells; and
wherein said sperm cells that have not undergone said acrosome reaction comprise non-acrosome-reacted sperm cells.

41. The method of claim 40, further comprising employing an acrosome reaction indicator which is associated with said acrosome-reacted sperm cells to differentiate said acrosome-reacted sperm cells from said non-acrosome-reacted sperm cells.

42. The method of claim 41, furthering comprising associating an acrosome reaction indicator associator with said acrosome reaction indicator to differentiate said acrosome-reacted sperm cells from said non-acrosome-reacted sperm cells.

43. The method of claim 42, wherein said acrosome reaction indicator comprises equatorin.

44. (canceled)

45. The method of claim 43, wherein said acrosome reaction indicator associator comprises an antibody which associates with said equatorin.

46-47. (canceled)

48. The method of claim 42, further comprising a first particle associated with said capacitation indicator associator, said first particle separable from said sample sperm cell population.

49. The method of claim 48, wherein said first particle is separable from said sample sperm cell population by the application of a force to which said first particle is responsive.

50. The method of claim 49, further comprising applying said force to said sample sperm cell population.

51. The method of claim 50, wherein said force comprises a centrifugal force, an electrostatic force, a gravitational force, a magnetic force, or the like, or combinations thereof.

52-60. (canceled)

61. The method of claim 49, wherein said capacitation indicator associator is conjugated to said first particle to provide a capacitation indicator associator particle.

62-79. (canceled)

80. The method of claim 61, further comprising second particle associated with said acrosome reaction indicator associator, said second particle separable from said sample sperm cell population:
wherein said acrosome reaction indicator associator is conjugated to said second particle to provide an acrosome reaction indicator associator particle.

81-97. (canceled)

98. The method of claim 80, further comprising:
differentiating between non-viable sperm cells and viable sperm cells; and
separating said non-viable sperm cells and said viable sperm cells.

99-128. (canceled)
